(19) United States
(12) Patent Application Publication
(10) Pub. No.: US 2024/0293349 A1
(43) Pub. Date: Sep. 5, 2024

(54) USE OF AN RXR AGONIST IN TREATING DRUG RESISTANT HER2+ CANCERS

(71) Applicants: Io Therapeutics, Inc., Spring, TX (US); Board of Regents, The University of Texas System, Austin, TX (US)

(72) Inventors: Martin E. Sanders, Spring, TX (US); Powel H. Brown, Houston, TX (US); Cassandra Moyer, Houston, TX (US); Abhijit Mazumdar, Houston, TX (US)

(21) Appl. No.: 18/649,476

(22) Filed: Apr. 29, 2024

Related U.S. Application Data
(63) Continuation of application No. 18/076,997, filed on Dec. 7, 2022, now Pat. No. 11,998,521.

(51) Int. Cl. A61K 31/216; A61K 39/00; A61P 35/00; C07K 16/32

(52) U.S. Cl. CPC A61K 31/216; A61P 35/00; C07K 16/32; A61K 2039/505

(57) ABSTRACT

The present specification provides combinations of active agents for the improved treatment of Her2⁺ cancers and associated methods of treatments. The combinations comprise an RXR agonist and a Her2-targeted therapeutic agent and may optionally further comprise thyroid hormone.

### CROSS-REFERENCE TO RELATED APPLICATIONS

The present application is a continuation of U.S. patent application Ser. No. 18/076,997 filed Dec. 7, 2022, which claims the benefit of U.S. Provisional Application No. 63/286,981 filed Dec. 7, 2021, the contents of each of which are incorporated by reference herein in their entirety.

### STATEMENT REGARDING FEDERALLY-SPONSORED RESEARCH

This invention was made with government support under grant number HHSN2612015000181 awarded by the National Institutes of Health. The government has certain rights in the invention.

### BACKGROUND

Compounds which have retinoid-like biological activity are well known in the art and are described in numerous United States patents including, but not limited to, U.S. Pat. Nos. 5,466,861; 5,675,033 and 5,917,082, all of which are herein incorporated by reference. Preclinical studies suggest that selective activation of Retinoid X Receptors (RXR), which modulate functions associated with differentiation, inhibition of cell growth, apoptosis and metastasis, with RXR-acting agents (rexinoids) may be useful in treating a variety of diseases associated with the biochemical functions modulated by RXR.

For example, TARGRETIN® (bexarotene), which is a RXR agonist having retinoic acid receptor (RAR) agonist activity as well, was approved by the U.S. Food and Drug Administration for the treatment, both oral and topical, of cutaneous manifestations of cutaneous T cell lymphoma in patients who are refractory to at least one prior systemic therapy. Encouraging results were obtained with TARGRETIN® in several Phase II studies in NSCLC (non-small cell lung cancer). However, the pivotal Phase III clinical study did not show increased survival. One possible explanation for the limited success of bexarotene is that its activation of RAR decreases its efficacy as an anticancer agent. Thus, more selective RXR agonists may hold greater promise.

Agents targeting human epidermal growth factor receptor 2 (Her2), both anti-Her2 antibodies and inhibitors of the tyrosine kinase activity of Her2, have had significant, but not universal, success in treating Her2⁺ cancers, particularly Her2⁺ breast cancers.

Treatments for cancer are ever evolving, gaining in specificity and sophistication. Early non-surgical cancer treatments generally targeted rapidly dividing cells which were more sensitive to radiological and chemical assault. Over time, more specific and less generally toxic treatments have been developed. Some treatments appear to have broad applicability, for example immune checkpoint inhibitors or rexinoids. Others are targeted to cancers that express a particular antigen or other biomarker involved in the regulation of proliferation or differentiation; including many monoclonal antibodies and kinase inhibitors. Yet as the variety of cancer treatments has grown, it has become ever harder to determine which treatments might be productively combined and for what indications.

### SUMMARY

Herein disclosed are improved methods of treatment of Her2⁺ cancers, particularly Her2⁺ cancers that are resistant to a Her2-targeted therapeutic agent. These methods comprise treating a patient having a Her2⁺ tumor with a combination of a Her2-targeting therapeutic agent and a RXR agonist capable of inhibiting cancer growth. In some embodiments, the cancer is also resistant to the RXR agonist. In some embodiments the treatment combination further comprises thyroid hormone.

In some embodiments, the RXR agonist is capable of activating RXR/Nurr1 heterodimeric receptors. In some embodiments the RXR agonist is a compound of Formula I as disclosed herein below, or a pharmaceutically-acceptable salt thereof. In some embodiments the RXR agonist is a compound of Formula II as disclosed herein below, or a pharmaceutically-acceptable salt thereof. In some embodiments, compounds of Formula I and Formula II, and their pharmaceutically-acceptable salts, are referred to as means for activating RXR/Nurr1 heterodimeric receptors or rexinoid means for inhibiting tumor growth.

In some embodiments, Her2-targeting therapeutic agent is an inhibitor of Her2 kinase activity or Her2-mediated signaling. In some embodiments Her2-targeting therapeutic agent is therapeutic anti-Her2 antibody. Therapeutic antibodies may mediate antibody-dependent cellular cytotoxicity (ADCC) instead of, or in addition to, inhibiting signaling (kinase activity). Trastuzumab and pertuzumab are examples of therapeutic anti-Her2 antibodies, as disclosed herein below. In some embodiments such antibodies are referred to as immunoglobulin means for inhibiting Her2⁺ tumor cell proliferation, means for mediating ADCC of Her2⁺ tumor cells, or immunoglobulin means for inhibiting Her2 signaling.

In some embodiments, a Her2-targeting therapeutic agent is an antibody-drug conjugate comprising an anti-Her2 antibody. In some embodiments, the anti-Her2 antibody has therapeutic activity alone, while in other embodiments it does not, merely serving to deliver a cytotoxic agent to Her2⁺ cells. Ado-trastuzumab emtansine is an example of a Her2-targeting antibody-drug conjugate, as disclosed herein below. In some embodiments such antibody-drug conjugates are referred to as means for delivering a cytotoxic agent to Her2⁺ cells.

In some embodiments, the inhibitor of Her2 kinase activity or Her2-mediated signaling is a small organic molecule (small drug) inhibitor of Her2 kinase activity. Lapatinib, neratinib, and tucatinib are examples of Her2 kinase inhibitors as disclosed herein below. In some embodiments such small drug inhibitor of Her2 kinase activity are referred to as small molecule means for inhibiting Her2 kinase activity.

In some embodiments, the Her2⁺ cancer is a Her2⁺ breast cancer. In some embodiments the Her2⁺ cancer is a Her2⁺ ovarian cancer, stomach cancer, adenocarcinoma of the lung, uterine cancer (such as serous endometrial carcinoma), gastric cancer, or salivary duct carcinoma.

In some embodiments, tumor cell growth is inhibited by the combination of the Her2-targeted therapeutic and the RXR agonist, and the inhibition of tumor cell growth by the combination is greater than additive inhibitory effects of each of the Her2-targeted therapeutic and the RXR agonist alone.

In some embodiments, the herein disclosed treatments are carried out concurrently with other pharmaceutical therapies or radiotherapies. In alternative embodiments, the herein disclosed treatments are the exclusive therapy in the time interval in which they are conducted. In some embodiments, the herein disclosed treatments serve as a debulking treatment in preparation for subsequent surgical removal of tumor. In some embodiments, the herein disclosed treatments are applied as adjuvant therapy subsequent to surgical removal of tumor to address any residual disease or potential recurrent disease.

Other embodiments include combination drug compositions or formulations comprising at least one Her2-targeting therapeutic agent and at least one RXR agonist capable of inhibiting cancer growth. In other embodiments the combination (used for treatment) may include more than one agent in one or the other classification (Her2 targeting agent and/or RXR agonist). In still other embodiments the combination may further include thyroid hormone. In some embodiments the thyroid hormone is thyroxine.

### BRIEF DESCRIPTION OF DRAWINGS

FIGS. 1A-B are three-dimensional plots depicting the growth inhibitory effects of IRX4204 and trastuzumab, alone and in combination, on breast cancer cell lines: MCF7 cells (FIG. 1A) and SkBr3 cells (FIG. 1B).

FIGS. 2A-D are three-dimensional plots depicting the growth inhibitory effects of IRX4204 and lapatinib, alone and in combination, on breast cancer cell lines: MCF7 cells (FIG. 2A), SkBr3 cells (FIG. 2B), BT474 cells (FIG. 2C), and MDA-MB-361 cells (FIG. 2D).

FIGS. 3A-D are three-dimensional plots depicting the growth inhibitory effect of IRX4204 and neratinib, alone and in combination, on breast cancer cell lines: MCF7 cells (FIG. 3A), SkBr3 cells (FIG. 3B), BT474 cells (FIG. 3C), and MDA-MB-361 cells (FIG. 3D).

FIGS. 4A-B depict the growth inhibitory effect of IRX4204 and neratinib, alone and in combination, on Her2⁺ breast cancer cell line HCC1954, which is resistant to both agents individually. FIG. 4A is a three-dimensional plot showing the growth inhibitory effect for a matrix of various concentrations of the two agents. FIG. 4B presents linear plots of the same data for no and 1000 nM IRX4204 for the various concentrations of neratinib.

FIG. 5A-B are three-dimensional plots depicting the growth inhibitory effect of IRX4204 and tucatinib (FIG. 5A) or lapatinib (FIG. 5B) alone and in combination, on Her2⁺ breast cancer cell line HCC1954, which is resistant to each of the agents individually.

### DESCRIPTION

The herein disclosed embodiments include methods of treating Her2⁺ cancers that are resistant to Her2-targeted therapeutic agents, such as resistant Her2⁺ carcinoma of the breast, with a combination a retinoid X receptor (RXR) agonist (rexinoid) and a Her2-targeted anticancer agent. The Her2⁺ cancers resistant to Her2-targeted therapeutic agents may also be resistant to the RXR agonist. Some embodiments further comprise administration of thyroid hormone in conjunction with the RXR agonist. Embodiments include a RXR agonist for use in combination with a Her2-targeted anticancer agent, or a thyroid hormone and a Her2-targeted anticancer agent, in the treatment of Her2⁺ cancers resistant to Her2-targeted therapeutic agents. In some embodiments, the Her2⁺ cancer is Her2⁺ breast cancer.

Even in light of the usefulness of combinations of Her2-targeted therapeutic agents and the disclosed RXR agonists in treating Her2⁺ cancers that are sensitive to Her2-targeted therapeutic agents, it is remarkable that the combination of a Her2-targeted therapeutic agent and the disclosed RXR agonists can be effective against Her2⁺ cancers that are resistant to the Her2-targeted therapeutic agent. Moreover, at appropriate concentrations of the therapeutic agents, the effect of these combinations is more than additive (that is, synergistic). It is even more remarkable that this holds true when the cancer is also resistant to the RXR agonist.

Three small molecule drugs that inhibit Her2 kinase activity, but are not close structural analogs of each other, neratinib, lapatinib, and tucatinib, were tested for growth inhibitory effect on a Her2⁺ breast cancer cell line in combination with the RXR agonist IRX4204. Synergistic growth inhibition was observed for all three kinase inhibitors with IRX4204. Thus, there is an apparent class effect with respect to inhibitors of Her2 kinase activity. In some embodiments, the inhibitor of Her2 kinase activity is a small organic molecule. In some embodiments, the inhibitor of Her2 kinase activity is an antibody, for example, trastuzumab (HERCEPTIN®), margetuximab (MARGENZA®), or pertuzumab (PERJETA®).

The methods of treatment disclosed herein involve the administration of a combination of two, three, or more therapeutic agents. Moreover, the administration of one of these agents may be described as being done in coordination or conjunction with another of these agents. By such combination, or administration of in coordination or in conjunction with, it is meant that the manner of administration of each of these agents is such that the physiologic effects of the agents overlap in time. This does not require that the agents be contained in the same composition or formulation, nor that they be administered as separate compositions at the same time, by the same route of administration, or on the same schedule, though in some embodiments any of the foregoing may be the case. For example, while it is possible to administer RXR agonists, thyroid hormone, and Her2 kinase inhibitors on a daily schedule, antibodies are more typically administered at intervals measured in weeks.

Among the earlier successful targeted cancer therapies are those targeting human epidermal growth factor receptor 2 (Her2). Her2-targeted treatments include monoclonal antibodies (mAbs), such as trastuzumab, margetuximab, and pertuzumab, and inhibitors of Her2 kinase activity, such as lapatinib, neratinib, and afatinib (which all also inhibit epidermal growth factor receptor (EGFR)). The rexinoid IRX4204 has shown activity against a variety of cancers in model systems (see for example, U.S. Patent Pub. 2008-0300312, which is incorporated herein by reference in its entirety for all that it teaches about the use of RXR agonists for the treatment of cancer) especially when used in combination with thyroid hormone (see for example, U.S. Patent Pub. 2008-0300312, which is incorporated herein by reference in its entirety for all that it teaches about the use of RXR agonists for the treatment of cancer). It is disclosed herein that the combination of IRX4204 and a Her2-targeted therapeutic agent are particularly effective against Her2⁺ cancers, such as Her2⁺ breast cancer. Thus, various embodiments include combinations of IRX4204 and a Her2-targeted therapeutic agent, and methods of treatment involving administration of both IRX4204 and a Her2-targeted therapeutic agent. Some embodiments further include thyroid hormone or the administration of thyroid hormone. Other embodiments exclude thyroid hormone or the administration of thyroid hormone.

A Her2-targeted therapeutic agent as used herein is a therapeutic agent that inhibits growth of cancer cells by inhibiting Her2 function. Such agents include mAbs that bind to Her2, antibody-drug conjugates comprising such mAbs, and inhibitors of Her2 tyrosine kinase activity. Collectively, such agents can be referred to as means for therapeutically targeting Her2. Some embodiments specifically include or are limited to one or more of these classes of agent, or one or more species within one or more of these classes of agent. Some embodiments specifically exclude one or more of these classes of agent, or one or more species within one or more of these classes of agent. Thus, means for therapeutically targeting Her2 are mAbs that bind to Her2, antibody-drug conjugates comprising mAbs that bind to Her2, and inhibitors of Her2 tyrosine kinase activity. In some embodiments, the mAb in an antibody-drug conjugate has therapeutic activity by itself, in other embodiments it does not.

Many embodiments comprise administration of a single anti-Her2 mAb, but some embodiments comprise administration of multiple anti-Her2 mAbs, for example, trastuzumab and pertuzumab. While the disclosed embodiments are generally described as using anti-Her2 mAbs, further embodiments can substitute anti-Her2 polyclonal antiserum for anti-Her2 mAb. Some embodiments can comprise administration of additional mAbs targeting other antigens, some embodiments specifically exclude administration of other antibodies.

### Anti-Her2 Antibodies

Her2 is a growth factor receptor found on, and implicated in, a variety of cancers, especially breast cancer but also, for example, gastroesophageal cancer, ovarian cancer, stomach cancer, adenocarcinoma of the lung, uterine cancer (such as serous endometrial carcinoma), or salivary duct carcinoma. Anti-Her2 antibodies are believed to work as anticancer agents through a combination of mechanisms including, but not limited to, inhibition of signaling through Her2, antibody-dependent cellular cytotoxicity (ADCC), and mediating presentation of tumor antigen by antigen presenting cells (APC), such as macrophages; additional mechanisms may also exist. The best understood and most clinically advanced anti-Her2 antibodies are trastuzumab, pertuzumab, and margetuximab, although the presently disclosed methods are not limited to these anti-Her2 mAbs.

In some embodiments, these antibodies are administered by intravenous infusion. In exemplary embodiments infusions may be done over 30-90 minutes and may occur at intervals of 1-3 weeks for as long as a year. In some such embodiments, the antibody is administered at an initial higher dose and a subsequent lower dose. In some such embodiments, the initial higher dosage is twice the subsequent lower dosage. In some such embodiments, the initial dose is single administration. In other such embodiments the initial dosage is administered multiple times before switching to the lower subsequent dosage. Specific examples of dosages and dose regimens can be found in the prescribing information for HERCEPTIN® and PERJETA®, which are incorporated herein by reference in their entireties.

Trastuzumab binds to domain IV of the extracellular segment of Her2. Trastuzumab inhibits the proliferation of cells that overexpress Her2 and mediates ADCC. Biosimilar antibodies to trastuzumab have been developed and are marketed in some jurisdictions.

Pertuzumab recognizes the extracellular dimerization domain (domain II) of Her2, a different epitope than trastuzumab. By preventing ligand-dependent dimerization, it inhibits signaling through Her2, leading to cell growth arrest and apoptosis. Pertuzumab also mediates ADCC. Pertuzumab augments the activity of trastuzumab in tumor xenograft models that over express Her2.

Margetuximab recognizes the same epitope as trastuzumab, but possesses an engineered Fc region designed to increase Fc-dependent mechanisms of immune attack, such as ADCC. The engineered Fc region confers increased binding to activating Fc-receptors (CD16A) and reduced binding to inhibitory Fc-receptors (CD16B) on immune effector cells, including monocytes, macrophages, dendritic cells and natural killer (NK) cells.

Further anti-Her2 mAbs include TrasGEX®, HM2, hertuzumab, and HT-19. TrasGEX® and HM2 are being developed as “biobetters” of trastuzumab, while the other two are independently derived. In HM2, a metal-binding motif has been incorporated into trastuzumab to aid conjugation. TrasGEX® is a glycosylation-optimized version of trastuzumab. Hertuzumab had a higher ELISA-based affinity for Her2 than trastuzumab. HT-19 is an IgG1 antibody that is non-competitive for HER2 binding with trastuzumab and pertuzumab; that is, it binds a different epitope than either of those two mAbs.

Many embodiments comprise administration of a single anti-Her2 mAb, but some embodiments comprise administration of multiple anti-Her2 mAbs, for example, trastuzumab and pertuzumab. While the disclosed embodiments are generally described as using anti-Her2 mAbs, further embodiments can substitute anti-Her2 polyclonal antiserum for anti-Her2 mAb.

### Antibody-Drug Conjugates

In addition to use of anti-Her2 mAbs themselves as therapeutic agents, anti-Her2 mAbs have also been incorporated into antibody-drug conjugates. One example is ado-trastuzumab emtansine (KADCYLA®). Further examples include A166, ALT-P7 (trastuzumab biobetter HM2 conjugated in a site-specific manner to monomethyl auristatin E), ARX788 (a monoclonal HER2 targeting antibody site-specifically conjugated, via a non-natural amino acid linker para-acetyl-phenylalanine (pAcF), to monomethyl auristatin F), DHES0815A (a monoclonal HER-2 targeting antibody linked to pyrrolo[2,1-c][1,4]benzodiazepine monoamide), DS-8201a (trastuzumab deruxtecan; trastuzumab, an enzymatically cleavable maleimide glycynglycyn-phenylalanyn-glycyn (GGFG) peptide linker and a topoisomerase I inhibitor, fam-trastuzumab deruxtecan (ENHERTU®), RC48 (humanized anti-HER2 antibody hertuzumab conjugated with monomethyl auristatin E (MMAE) via a cleavable linker), SYD985 (([vic-]trastuzumab duocarmazine; trastuzumab linked via a cleavable valine-citrulline peptide to the synthetic duocarmycin analog seco-duocarmycin-hydroxybenzamide azaindole), MEDI4276 (HER2-bispecific antibody targeting two different epitopes on HER2, site-specifically conjugated via a maleimidocaproyl linker to the potent tubulysin-based microtubule inhibitor AZ13599185) and XMT-1522 (TAK-522; HT-19 conjugated with the DOLAFLEXIN® platform to auristatin F-hydroxypropylamide).

In some embodiments, the Her2-targeting component comprises, or is, an antibody-drug conjugate comprising an anti-Her2 antibody.

### Her2 Kinase Inhibitors

Her2 and EGFR are closely related protein tyrosine kinases and many drugs developed as an inhibitor of one also inhibit the other. At least four drugs that are irreversible inhibitors of these kinases are now marketed as a cancer treatment (lapatinib, neratinib, afatinib, and dacomitinib), though the indications vary. It should be noted that not all EGFR inhibitors are irreversible inhibitors or are known to cross-inhibit Her2. EGFR inhibitors that do not—or are not known to—inhibit Her2 should not be considered Her2 inhibitors as the term is used herein. In some embodiments, the Her2 inhibitor is an irreversible inhibitor. In some embodiments, the Her2 inhibitor is not a reversible inhibitor. In some embodiments, the Her2 inhibitor is a reversible inhibitor.

Lapatinib (TYKERB®) (CAS No. 231277-92-2), typically provided as Lapatinib Ditosylate (CAS No. 388082-77-7) or the monohydrate thereof, can be administered, according to its prescribing instructions which are incorporated herein by reference in their entirety, on a 21 day treatment cycle, at 1250 or 1500 mg once daily, depending on indication. Lapatinib plus capecitabine is taken on days 1 to 14. Lapatinib alone is taken on days 15 to 21. At the end of the 21 days, the treatment cycle should be repeated until disease progression or unacceptable toxicity occurs. Capecitabine is administered orally in two doses approximately 12 hours apart at a dosage of 2000 mg/m²/day.

Neratinib (NERLYNX®) (CAS No. 698387-09-6) can be administered, according to its prescribing instructions which are incorporated herein by reference in their entirety, with food at an initial dose of 240 mg/day and taken daily for a year. If toxicity exceeds grade 1, the dose can be reduced by 40 mg/day in stepwise fashion until toxicity is grade 1 or less. If the dose has been reduced to 120 mg/day and toxicity remains greater than grade 1, treatment with Neratinib should be discontinued.

Afatinib (GILOTRIF®) (CAS No. 850140-72-6) can be administered, according to its prescribing instructions which are incorporated herein by reference in their entirety, orally without food once daily at 40 mg/day until disease progression or no longer tolerated by the patient.

Dacomitinib (VIZIMPRO®) (CAS No. 1110813-31-4) can be administered, according to its prescribing instructions which are incorporated herein by reference in their entirety, orally with or without food, once daily at 45 mg/day until disease progression or unacceptable toxicity occurs. Upon occurrence of unacceptable toxicity, the dosage can be reduced in stepwise fashion to 30 or 15 mg/day.

Tucatinib (TUKYSA®) (CAS No. 937263-43-9) can be administered, according to its prescribing instructions which are incorporated herein by reference in their entirety, orally with or without food, 300 mg twice daily (12 hours apart) in combination with trastuzumab and capecitabine until disease progression or unacceptable toxicity. Upon occurrence of unacceptable toxicity, the dosage can be reduced in stepwise fashion, 50 mg per step, to 150 mg, twice daily.

The above dosing information, in addition to disclosing specific embodiments in which these Her2 kinase inhibitors may be used in combination with an RXR agonist, provides general guidance as to dosing practices with such drugs. The disclosed embodiments are not necessarily limited to these specific dosing regimens and it is within the skill of the physician to modify these regimens for individual patients. Due to the improved and synergistic effect of these drugs when used in combination with a RXR agonist, unacceptable toxicity may be avoided by use of lower dosages of the kinase inhibitor while still achieve beneficial therapeutic effect.

Other Her2 kinase inhibitors include canertinib (CAS No. 267243-28-7), sapitinib (CAS No. 848942-61-0), CP-724714 (CAS No. 537705-08-1), and CUDC-101 (CAS No. 1012054-59-9).

### RXR Agonists

Preclinical studies with rexinoids suggest that selective activation of Retinoid X Receptors (RXR), which modulate functions associated with differentiation, inhibition of cell growth, apoptosis and metastasis, may be useful in treating a variety of diseases associated with the biochemical functions modulated by RXR.

The Retinoic Acid Receptors (RARs) and RXRs and their cognate ligands function by distinct mechanisms. The term “RAR” as used herein refers to one or more of RARα, RARβ, or RARγ. The term “RXR” as used herein refers to one or more of RXRα, RXRβ, or RXRγ. A RAR biomarker is a distinctive biological, biochemical or biologically derived indicator that signifies patient RAR activity. RAR biomarkers include, but are not limited to, CYP26 levels, CRBPI levels, and the like, and combinations thereof.

In some embodiments, the RAR activation threshold means one or more of a CYP26 level which is 25% increased over baseline and a CRBPI level 25% increased over baseline. The RARs form heterodimers with RXRs and these RAR/RXR heterodimers bind to specific response elements in the promoter regions of target genes. The binding of RAR agonists to the RAR receptor of the heterodimer results in activation of transcription of target genes leading to retinoid effects. The disclosed RXR agonists do not activate RAR/RXR heterodimers. RXR heterodimer complexes like RAR/RXR can be referred to as non-permissive RXR heterodimers as activation of transcription due to ligand-binding occurs only at the non-RXR protein (e.g., RAR); activation of transcription does not occur due to ligand binding at the RXR.

RXRs also interact with nuclear receptors other than RARs and RXR agonists and may elicit some of their biological effects by binding to such RXR/receptor complexes. These RXR/receptor complexes can be referred to as permissive RXR heterodimers as activation of transcription due to ligand-binding could occur at the RXR, the other receptor, or both receptors. Examples of permissive RXR heterodimers include, without limitation, peroxisome proliferator activated receptor/RXR (PPAR/RXR), farnesyl X receptor/RXR (FXR/RXR), nuclear receptor related-1 protein (Nurr1/RXR) and liver X receptor/RXR (LXR/RXR). Alternately, RXRs may form RXR/RXR homodimers which can be activated by RXR agonists leading to rexinoid effects. Also, RXRs interact with proteins other than nuclear receptors and ligand binding to an RXR within such protein complexes can also lead to rexinoid effects. Due to these differences in mechanisms of action, RXR agonists and RAR agonists elicit distinct biological outcomes and even in the instances where they mediate similar biological effects, they do so by different mechanisms. Moreover, the unwanted side effects of retinoids, such as pro-inflammatory responses or mucocutaneous toxicity, are mediated by activation of one or more of the RAR receptor subtypes. Stated another way, biological effects mediated via RXR pathways would not induce pro-inflammatory responses, and thus, would not result in unwanted side effects.

Thus, aspects of the present specification provide, in part, a RXR agonist. As used herein, the term “RXR agonist”, is synonymous with “selective RXR agonist” and refers to a compound that selectively binds to one or more RXR receptors like a RXRα, a RXRβ, or a RXRγ in a manner that elicits gene transcription via an RXR response element. As used herein, the term “selectively binds,” when made in reference to a RXR agonist, refers to the discriminatory binding of a RXR agonist to the indicated target receptor like a RXRα, a RXRβ, or a RXRγ such that the RXR agonist does not substantially bind with non-target receptors like a RARα, a RARβ or a RARγ. In some embodiments, the term “RXR agonist” includes esters of RXR agonists.

For each of the herein disclosed embodiments, the RXR agonists can be compounds having the structure of Formula I

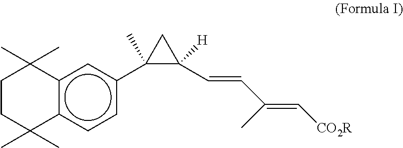

wherein R is H, or lower alkyl of 1 to 6 carbons, or a pharmaceutically acceptable salt thereof.

Also disclosed herein are esters of RXR agonists. An ester may be derived from a carboxylic acid of C1, or an ester may be derived from a carboxylic acid functional group on another part of the molecule, such as on a phenyl ring. While not intending to be limiting, an ester may be an alkyl ester, an aryl ester, or a heteroaryl ester. The term alkyl has the meaning generally understood by those skilled in the art and refers to linear, branched, or cyclic alkyl moieties. C₁₋₆ alkyl esters are particularly useful, where alkyl part of the ester has from 1 to 6 carbon atoms and includes, but is not limited to, methyl, ethyl, propyl, isopropyl, n-butyl, sec-butyl, iso-butyl, t-butyl, pentyl isomers, hexyl isomers, cyclopropyl, cyclobutyl, cyclopentyl, cyclohexyl, and combinations thereof having from 1-6 carbon atoms, etc. In some embodiments the RXR agonist is the ethyl ester of formula I.

In some embodiments the RXR agonist is 3,7-dimethyl-6(S),7(S)-methano,7-[1,1,4,4-tetramethyl-1,2,3,4-tetrahydron-aphth-7-yl]2(E), 4(E) heptadienoic acid, also known as IRX4204, and has the following chemical structure:

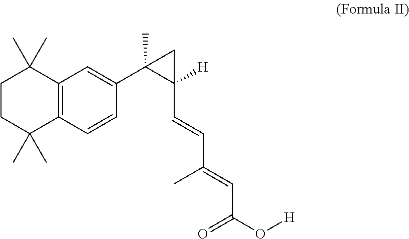 [SMILES: CC(/C=C/[C@@H]1C[C@]1(C)c1ccc2c(c1)C(C)(C)CCC2(C)C)=C\C(=O)O]

Pharmaceutically acceptable salts of RXR agonists can also be used in the disclosed embodiments. Compounds disclosed herein which possess a sufficiently acidic, a sufficiently basic, or both functional groups, and accordingly can react with any of a number of organic or inorganic bases, and inorganic and organic acids, to form a salt.

Acids commonly employed to form acid addition salts from RXR agonists with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Bases commonly employed to form base addition salts from RXR agonists with acidic groups include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like.

IRX4204, like some other RXR ligands, does not activate non-permissive heterodimers such as RAR/RXR. However, IRX4204, is unique in that it specifically activates the Nurr1/RXR heterodimer and does not activate other permissive RXR heterodimers such as PPAR/RXR, FXR/RXR, and LXR/RXR. Other RXR ligands generally activate these permissive RXR heterodimers. Thus, all RXR ligands cannot be classified as belonging to one class. IRX4204 belongs to a unique class of RXR ligands which specifically activate RXR homodimers and only one of the permissive RXR heterodimers, namely the Nurr1/RXR heterodimer.

In one embodiment, the selective RXR agonist does not activate to any appreciable degree the permissive heterodimers PPAR/RXR, FXR/RXR, and LXR/RXR. In another embodiment, the selective RXR agonist, activates the permissive heterodimer Nurr1/RXR. One example of such a selective RXR agonist is 3,7-dimethyl-6(S),7(S)-methano,7-[1,1,4,4-tetramethyl-1,2,3,4-tetrahydronaphth-7-yl]2(E),4(E) heptadienoic acid (IRX4204) disclosed herein, the structure of which is shown in Formula II. In other aspects of this embodiment, the RXR agonists activates the permissive heterodimers PPAR/RXR, FXR/RXR, or LXR/RXR by 1% or less, 2% or less, 3% or less, 4% or less, 5% or less, 6% or less, 7% or less, 8% or less, 9% or less, or 10% or less relative to the ability of activating agonists to the non-RXR receptor to activate the same permissive heterodimer. Examples of RXR agonists, which activates one or more of PPAR/RXR, FXR/RXR, or LXR/RXR include LGD1069 (bexarotene) and LGD268.

Binding specificity is the ability of a RXR agonist to discriminate between a RXR receptor and a receptor that does not contain its binding site, such as a RAR receptor.

Particular embodiments provide methods of treating cancer comprising administering to a patient in need of such treatment a RXR agonist at a level below an RAR activating threshold and at or above an RXR activating threshold.

For IRX4204, the RAR EC₁₀ (the concentration effective to cause a 10% of maximal activation of the RAR) is 300 nM for the □□ isoform and 200 nM for the □□ and □ isoforms. Thus, in some embodiments, concentrations not exceeding 200 nM are considered to be below an RAR activating concentration. For IRX4204, the RXR EC₉₀ (the concentration effective to cause a 90% of maximal activation of the RXR) is 0.1 nM for the □□ and □□ isoforms and 1 nM for the □□ isoform. Thus, in some embodiments concentrations of at least 0.1 nM are considered to be above an RXR activating threshold. Based on studies in humans, oral dosages of IRX4204 of 20 mg/m²/day will produce systemic concentrations that remain below 200 nM. Similarly, it is estimated that an oral dosage in the range of 0.01 to 0.02 mg/m²/day will produce systemic concentrations of 0.1 nM or greater. Thus, in various individual embodiments a dosage of IRX4204 is at least 0.01, 0.02, 0.03, 0.05, 0.1, 0.3, 0.5, 1, 3 or 5 mg/m²/day and does not exceed 150, 200, or 300 mg/m²/day, or any range bound by a pair of these values.

In other embodiments, the dosage for a human adult of the RXR agonist, for example IRX4204, is from 0.2 to 300 mg/day, such as in individual embodiments, from 0.5, 1, 2, 3, 4, 5, 6, 7, 8, 9, or 10 mg/day, but not to exceed 10, 15, 20, 50, or 100 mg/day, or any range bound by a pair of these values.

The RXR agonist can be administered to a mammal using standard administration techniques, including parenteral, oral, intravenous, intraperitoneal, subcutaneous, pulmonary, transdermal, intramuscular, intranasal, buccal, sublingual, or suppository administration. The term “parenteral,” as used herein, includes intravenous, intramuscular, subcutaneous, rectal, vaginal, and intraperitoneal administration. The RXR agonist preferably is suitable for oral administration, for example as a pill, tablet or capsule. Administration may be continuous or intermittent. In certain embodiments, the total daily dosage of RXR agonist can be administered as a single dose or as two doses administered with a 24 hour period spaced 8 to 16, or 10 to 14, hours apart.

### Thyroid Hormone

Both biologically sourced and synthetic thyroid hormones have been used in medicine. The major forms of thyroid hormone are referred to as T₃ (triiodothyronine) and T₄ (thyroxine). Thyroxine is less active but has a longer half-life and is sometimes considered a prohormone of triiodothyronine. As used herein, the term “thyroid hormone” refers to both thyroxine and triiodothyronine. Thyroxine (thyroid hormone T₄, levothyroxine sodium) is a tyrosine-based hormone produced by the thyroid gland and is primarily responsible for regulation of metabolism. Both have wide commercial availability and are suitable for use in the herein disclosed embodiments. However, the synthetic form of T₄, levothyroxine, is much more commonly utilized clinically (except in patients unable to convert T₄ into T₃) as its longer half-life in the body facilitates once-daily administration. In some embodiments, the administered thyroid hormone is specifically thyroxine. In some embodiments, the administered thyroid hormone is triiodothyronine.

Administration of RXR agonists, or esters thereof, may lead to the suppression of serum thyroid hormones and possibly to clinical hypothyroidism and related conditions. However, in some embodiments thyroid hormone is not co-administered (or is not primarily co-administered) to remediate a suppression of serum thyroid hormone levels. Co-administration of thyroid hormone with an RXR agonist improves the RXR agonist's anti-cancer efficacy, as compared to the effect of the RXR agonist alone, likely through multiple mechanisms of action. The co-administered thyroid hormone can also mitigate the hypothyroid-inducing effects of the RXR agonist, thereby improving the clinical safety and tolerability of the treatment. Thus, in preferred embodiments, thyroid hormone is co-administered with an RXR agonist to improve the efficacy of the treatment, whether or not administration of the RXR agonist has caused, or is expected to cause, clinical hypothyroidism. By administration of thyroid hormone in coordination or in conjunction with the RXR agonist it is meant that the manner of administration of each of these two agents is such that the physiologic effects of the two agents overlap in time. This does not require that the RXR agonist and thyroid hormone be contained in the same composition or formulation, or that they be administered as separate compositions at the same time, by the same route of administration, or on the same schedule, though in some embodiments any of the foregoing may be the case.

Suitable thyroxine doses are generally from about 5 μg/day to about 250 μg/day orally initially with an increase in dose every 2-4 weeks as needed. In other embodiments, the suitable thyroxine dose is from about 5 μg/day to about 225 μg/day, from about 7.5 μg/day to about 200 μg/day, from about 10 μg/day to about 175 μg/day, from about 12.5 μg/day to about 150 μg/day, from about 15 μg/day to about 125 μg/day, from about 17.5 μg/day to about 100 μg/day, from about 20 μg/day to about 100 μg/day, from about 22.5 μg/day to about 100 μg/day, from about 25 μg/day to about 100 μg/day, from about 5 μg/day to about 200 μg/day, from about 5 μg/day to about 100 μg/day, from about 7.5 μg/day to about 90 μg/day, from about 10 μg/day to about 80 μg/day, from about 12.5 μg/day to about 60 μg/day, or from about 15 μg/day to about 50 μg/day. Increases in dose are generally made in increments of about 5 μg/day, about 7.5 μg/day, about 10 μg/day, about 12.5 μg/day, about 15 μg/day, about 20 μg/day, or about 25 μg/day. In certain embodiments, the suitable thyroid hormone dose is a dose able to produce serum levels of T₄ in the top 50%, the top 60%, the top 70%, the top 80%, or the top 90% of the normal range for the testing laboratory. As the normal range of T₄ levels may vary by testing laboratory, the target T₄ levels are based on normal ranges determined for each particular testing laboratory.

For each embodiment involving a combination of RXR agonist and Her2-targeting therapeutic agent, there is a parallel embodiment in which the combination further comprises thyroid hormone.

### Pharmaceutical Compositions and Formulations

The various component active agents used in the herein described treatments will typically exist as pharmaceutical compositions or formulations. Such compositions or formulations may be a liquid formulation, semi-solid formulation, or a solid formulation. A formulation disclosed herein can be produced in a manner to form one phase, such as, e.g., an oil or a solid. Alternatively, a formulation disclosed herein can be produced in a manner to form two phases, such as, e.g., an emulsion. A pharmaceutical composition disclosed herein intended for such administration may be prepared according to any method known to the art for the manufacture of pharmaceutical compositions.

Liquid formulations suitable for parenteral injection or for nasal sprays may comprise physiologically acceptable sterile aqueous or nonaqueous solutions, dispersions, suspensions or emulsions and sterile powders for reconstitution into sterile injectable solutions or dispersions. Formulations suitable for nasal administration may comprise physiologically acceptable sterile aqueous or nonaqueous solutions, dispersions, suspensions or emulsions. Examples of suitable aqueous and nonaqueous carriers, diluents, solvents or vehicles include water, ethanol, polyols (propylene glycol, polyethyleneglycol (PEG), glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

A pharmaceutical composition disclosed herein can optionally include a pharmaceutically acceptable carrier that facilitates processing of an active compound into pharmaceutically acceptable compositions. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. This definition is also application to the phrase “pharmaceutically-acceptable salts”. As used herein, the term “pharmacologically acceptable carrier” is synonymous with “pharmacological carrier” and refers to any carrier that has substantially no long term or permanent detrimental effect when administered and encompasses terms such as “pharmacologically acceptable vehicle, stabilizer, diluent, additive, auxiliary, or excipient.” Such a carrier generally is mixed with an active compound or permitted to dilute or enclose the active compound and can be a solid, semi-solid, or liquid agent. It is understood that the active compounds can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., starch, magnesium stearate, mannitol, sodium saccharin, talcum, cellulose, glucose, sucrose, lactose, trehalose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active compound, its use in pharmaceutically acceptable compositions is contemplated. Non-limiting examples of specific uses of such pharmaceutical carriers can be found in Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003). These protocols are routine and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, borate buffers, citrate buffers, phosphate buffers, neutral buffered saline, and phosphate buffered saline. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole, and butylated hydroxytoluene. Useful preservatives include, without limitation, benzalkonium chloride, chlorobutanol, thimerosal, phenylmercuric acetate, phenylmercuric nitrate, a stabilized oxy chloro composition, such as, e.g., sodium chlorite and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition useful in the invention.

Pharmaceutical formulations suitable for administration by inhalation include fine particle dusts or mists, which may be generated by means of various types of metered, dose pressurized aerosols, nebulizers, or insufflators.

Semi-solid formulations suitable for topical administration include, without limitation, ointments, creams, salves, and gels. In such solid formulations, the active compound may be admixed with at least one inert customary excipient (or carrier) such as, a lipid and/or polyethylene glycol.

Solid formulations suitable for oral administration include capsules, tablets, pills, powders and granules. In such solid formulations, the active compound may be admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example, paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

The small molecule components of the various embodiments, that is, the RXR agonist, thyroid hormone, and Her2 kinase inhibitors are capable of being formulated in solid, oral dosage forms. The antibody components are generally formulated as liquids typically for intravenous infusion. In alternative embodiments the antibody components may be supplied in lyophilized form for reconstitution as a liquid locally at the site of treatment, where they are also typically infused intravenously into the patient. While intravenous infusion is typical, in alternative embodiments the antibody may be administered by another route of administration, such as subcutaneous injection or infusion.

### Treatment

As used herein, the terms “treatment,” “treating,” and the like refer to obtaining a desired pharmacologic and/or physiologic effect. This may be observed directly as a slowing of tumor growth, stabilization of disease, or a partial or complete response (that is, tumor regression or elimination of tumors), or extended overall or disease-free survival. Treatment may also be observed as an amelioration or reduction of symptoms related to the underlying cancer. However, as cancer treatment, the disclosed embodiments' aim and mechanism is directed to inhibiting, stabilizing, or reducing tumor growth (including metastases), or partially or completely eliminating tumors, or extending overall or disease-free survival; effects on other cancer symptoms are secondary. Direct treatment of such other symptoms (for example, pain, nausea, loss of appetite, etc.) is not within the scope of treating cancer as used herein. That is, treating a symptom, for example, cachexia in a cancer patient is not treating cancer. However, an agent that treats cancer (e.g., has an impact on the growth and/or spread of cancer) may also ameliorate a symptom, such as cachexia, either indirectly, through its effect on the cancer, or directly, through a pleiotropic effect. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the Her2-targeted therapy, RXR agonist, and if used, thyroid hormone, to elicit a desired response in the individual.

However, the dose administered to a mammal, particularly a human, in the context of the present methods, should be sufficient to effect a therapeutic response in the mammal over a reasonable timeframe. One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the potency of the specific compound, the age, condition, body weight, sex and response of the patient to be treated, and the stage/severity of the disease.

Treatment activity includes the administration of the medicaments, dosage forms, and pharmaceutical compositions described herein to a patient, especially according to the various methods of treatment disclosed herein, whether by a healthcare professional, the patient his/herself, or any other person. Treatment activities include the orders, instructions, and advice of healthcare professionals such as physicians, physician's assistants, nurse practitioners, and the like that are then acted upon by any other person including other healthcare professionals or the patient his/herself. In some embodiments, treatment activity can also include encouraging, inducing, or mandating that a particular medicament, or combination thereof, be chosen for treatment of a condition—and the medicament is actually used—by approving insurance coverage for the medicament, denying coverage for an alternative medicament, including the medicament on, or excluding an alternative medicament, from a drug formulary, or offering a financial incentive to use the medicament, as might be done by an insurance company or a pharmacy benefits management company, and the like. In some embodiments, treatment activity can also include encouraging, inducing, or mandating that a particular medicament be chosen for treatment of a condition—and the medicament is actually used—by a policy or practice standard as might be established by a hospital, clinic, health maintenance organization, medical practice or physicians group, and the like.

To benefit from the combined effect of a RXR agonist of Formula I (or pharmaceutically acceptable salt or ester thereof) and Her2-targeted therapeutics, embodiments include methods of treatment comprising or consisting of administering RXR agonist of Formula I (or pharmaceutically acceptable salt thereof) and a Her2-targeted therapeutic to a patient having a Her2⁺ cancer. Some embodiments further comprise administration of thyroid hormone in coordination with administration of the RXR agonist. In some embodiments the Her2⁺ cancer is a Her2⁺ breast cancer. In some embodiments the Her2⁺ cancer is a Her2⁺ gastroesophageal cancer, ovarian cancer, stomach cancer, adenocarcinoma of the lung, uterine cancer (such as serous endometrial carcinoma), or salivary duct carcinoma. Some embodiments specifically include one or more of these cancers. Other embodiments specifically exclude one or more of these cancers.

In various embodiments the herein disclosed treatments may be applied as a primary therapy, as a debulking therapy prior to surgical removal of tumor, or as an adjuvant therapy subsequent to any mode of primary therapy (especially surgery) to address residual disease and/or lower the risk of recurrent cancer.

In some embodiments, the patient having a Her2⁺ cancer has not been previously treated with either RXR agonist of Formula I (or pharmaceutically acceptable salt thereof) or a Her2-targeted therapeutic. In some embodiments the patient has been previously treated with RXR agonist of Formula I (or pharmaceutically acceptable salt thereof) and has achieved stable disease or a partial response (in some embodiments, as defined by RECIST or iRECIST criteria)—that is, the cancer is sensitive to RXR agonist of Formula I (or pharmaceutically acceptable salt thereof)—and a Her2-targeted therapeutic is added to the treatment regimen. In some embodiments the patient has been previously treated with a Her2-targeted therapeutic and has achieved stable disease or a partial response (in some embodiments, as defined by RECIST or iRECIST criteria)—that is, the cancer is sensitive to a Her2-targeted therapeutic—and RXR agonist of Formula I (or pharmaceutically acceptable salt thereof) is added to the treatment regimen.

Thus, some embodiments comprise administration of an RXR agonist to a patient with a Her2⁺ tumor who has received, is receiving, or is scheduled to receive, a Her2-targeted therapeutic agent. Some embodiments comprise administration of an RXR agonist to a patient in whom a Her2-targeted therapeutic agent has had some therapeutic effect (less than a complete response), that is administration of the RXR agonist is added to the therapeutic regimen for the Her2-targeted therapeutic agent. Some embodiments comprise administration of a Her2-targeted therapeutic agent to a patient in whom an RXR agonist (or RXR agonist in conjunction with thyroid hormone) has had some therapeutic effect (less than a complete response), that is administration of the Her2-targeted therapeutic agent is added to the therapeutic regimen for the RXR agonist.

Therapeutic efficacy can be monitored by periodic assessment of treated patients. For repeated administrations over several days or longer, the treatment can be repeated until a desired suppression of disease or disease symptoms occurs. However, other dosage regimens may be useful and are within the scope of the present disclosure. Antibodies typically have a much longer half-life in the body than the other active agents used in these methods and therefore there will typically be substantially longer intervals (measured in weeks) between administrations.

The effectiveness of cancer therapy is typically measured in terms of “response.” The techniques to monitor responses can be similar to the tests used to diagnose cancer such as, but not limited to:
A lump or tumor involving some lymph nodes can be felt and measured externally by physical examination.
Some internal cancer tumors will show up on an x-ray or CT scan and can be measured with a ruler.
Blood tests, including those that measure organ function can be performed.
A tumor marker test can be done for certain cancers.

Regardless of the test used, whether blood test, cell count, or tumor marker test, it is repeated at specific intervals so that the results can be compared to earlier tests of the same type.

Response to cancer treatment is defined several ways:
Complete response—all of the cancer or tumor disappears; there is no evidence of disease. Expression level of tumor marker (if applicable) may fall within the normal range.
Partial response—the cancer has shrunk by a percentage but disease remains. Levels of a tumor marker (if applicable) may have fallen (or increased, based on the tumor marker, as an indication of decreased tumor burden) but evidence of disease remains.
Stable disease—the cancer has neither grown nor shrunk; the amount of disease has not changed. A tumor marker (if applicable) has not changed significantly.
Disease progression—the cancer has grown; there is more disease now than before treatment. A tumor marker test (if applicable) shows that a tumor marker has risen.

Other measures of the efficacy of cancer treatment include intervals of overall survival (that is time to death from any cause, measured from diagnosis or from initiation of the treatment being evaluated)), cancer-free survival (that is, the length of time after a complete response cancer remains undetectable), and progression-free survival (that is, the length of time after disease stabilization or partial response that resumed tumor growth is not detectable).

There are two standard methods for the evaluation of solid cancer treatment response with regard to tumor size (tumor burden), the WHO and RECIST standards. These methods measure a solid tumor to compare a current tumor with past measurements or to compare changes with future measurements and to make changes in a treatment regimen. In the WHO method, the solid tumor's long and short axes are measured with the product of these two measurements is then calculated; if there are multiple solid tumors, the sum of all the products is calculated. In the RECIST method, only the long axis is measured. If there are multiple solid tumors, the sum of all the long axes measurements is calculated. However, with lymph nodes, the short axis is measured instead of the long axis. There is also a variation of the RECIST method for immunotherapies (iRECIST) which takes into account distinctive behaviors linked to these types of therapeutics, such as delayed responses after pseudoprogression. Both the RECIST 1.1 guidelines and the iRecist guidelines are incorporated by reference herein in their entirety.

In some embodiments of the herein disclosed methods, the tumor burden of a treated patient is reduced by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In other embodiments, the 1-year survival rate of treated subjects is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In other embodiments, the 5-year survival rate of treated subjects is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In other embodiments, the 10-year survival rate of treated subjects is increased by about 5%, about 10%, about 15%, about 20%, about 25%, about 30%, about 35%, about 40%, about 45%, about 50%, about 55% about 60%, about 65%, about 70%, about 75%, about 80%, about 90%, about 95%, about 100%, or any range bound by these values.

In yet other embodiments, the subject has a sustained remission of at least 6 months, at least 7 months, at least 8 months, at least 9 months, at least 10 months, at least 11 months, at least 12 months, at least 14 months, at least 16 months, at least 18 months, at least 20 months, at least 22 months, at least 24 months, at least 27 months, at least 30 months, at least 33 months, at least 36 months, at least 42 months, at least 48 months, at least 54 months, or at least 60 months or more.

In other embodiments, the methods may additionally help to treat or alleviate conditions, symptoms, or disorders related to cancer. In some embodiments, these conditions or symptoms may include, but are not limited to, anemia, asthenia, cachexia, Cushing's syndrome, fatigue, gout, gum disease, hematuria, hypercalcemia, hypothyroidism, internal bleeding, hair loss, mesothelioma, nausea, night sweats, neutropenia, paraneoplastic syndromes, pleuritis, polymyalgia rheumatica, rhabdomyolysis, stress, swollen lymph nodes, thrombocytopenia, vitamin D deficiency, or weight loss. While a cancer treatment may reduce or treat associated symptoms, treating symptoms associated with cancer, is not treating cancer if there is no expectation that tumor will be reduced or eliminated or their growth or spread will be inhibited.

Toxicities and adverse events are sometimes graded according to a 5 point scale. A grade 1 or mild toxicity is asymptomatic or induces only mild symptoms; may be characterized by clinical or diagnostic observations only; and intervention is not indicated. A grade 2 or moderate toxicity may impair activities of daily living (such as preparing meals, shopping, managing money, using the telephone, etc.) but only minimal, local, or non-invasive interventions are indicated. Grade 3 toxicities are medically significant but not immediately life-threatening; hospitalization or prolongation of hospitalization is indicated; activities of daily living related to self-care (such as bathing, dressing and undressing, feeding oneself, using the toilet, taking medications, and not being bedridden) may be impaired. Grade 4 toxicities are life-threatening and urgent intervention is indicated. Grade 5 toxicity produces an adverse event-related death. Thus in various embodiments, use of an RXR agonist, or RXR agonist and thyroid hormone, reduces the grade of a toxicity that would otherwise be associated with use of the Her2-targeted therapy, by allowing a lower dose to be used without substantial sacrifice of efficacy. In some embodiments, use of an RXR agonist, or RXR agonist and thyroid hormone, in combination with the Her2-targeted therapeutic agent limits a toxicity to grade 1 or less, or produces no observation of the toxicity, without substantial reduction of efficacy as would be expected from the Her2-targeted therapeutic agent alone. In some embodiments, the combined use of the Her2-targeted therapeutic agent and the RXR agonist, or RXR agonist and thyroid hormone, allows continued use of the Her2-targeted therapeutic agent at a lower dosage with therapeutic effect in instances where treatment with the Her2-targeted therapeutic agent would have had to have been discontinued due to unacceptable toxicity. In some of these embodiments, the Her2-targeted therapeutic agent comprises a Her2 kinase inhibitor.

The combination of the disclosed RXR agonists and the Her2 targeted therapeutics are synergistic in effect. That is, they interact in a positive manner to produce a greater inhibition of tumor cell growth than would be expected from the independent (non-interacting) effects of the two. Thus, some embodiments produce improved efficacy. Other embodiments allow for the reduction of dosage in order to reduce toxicity while still achieving at least similar efficacy as provided by an individual therapeutic agent. In some embodiments, both reduced toxicity and improved efficacy (as compared to the more toxic single agent) is achieved.

For each method of treatment there are further parallel embodiments related to the foregoing methods directed to use of the RXR agonist in conjunction with a Her2-targeted therapeutic agent, or a Her2-targeted therapeutic agent and thyroid hormone, to treat Her2⁺ cancer; directed to use of the RXR agonist in the manufacture of a medicament for use in combination with a Her2-targeted therapeutic agent, or a Her2-targeted therapeutic agent and thyroid hormone, to treat Her2⁺ cancer.

Further embodiments include a combination comprising a RXR agonist as herein described and a Her2-targeted therapeutic agent. Some embodiments further comprise a thyroid hormone. In some embodiments, the Her2-targeted therapeutic agent is an anti-Her2 antibody. In some embodiments the Her2-targeted therapeutic agent is a Her2 kinase inhibitor.

Further embodiments include kits comprising the above combinations. The kits may additionally comprise solvents, diluents, injectors, and the like that may facilitate administration of one or more of the therapeutic agents. The kits may further comprise instructions for the coordinated use of the therapeutic agents utilized in the disclosed methods, whether or not any particular agent is supplied in the kit.

### LIST OF PARTICULAR EMBODIMENTS

The following listing of embodiments is illustrative of the variety of embodiments with respect to breadth, combinations and sub-combinations, class of invention, etc., elucidated herein, but is not intended to be an exhaustive enumeration of all embodiments finding support herein.

Embodiment 1. A method of treating a patient with Her2⁺ cancer resistant to a Her2-targeted therapeutic agent comprising administering a RXR agonist of Formula I,

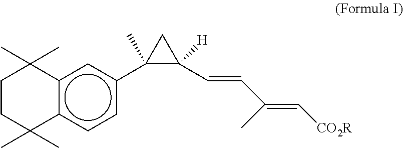

wherein R is H, or lower alkyl of 1 to 6 carbons; or a pharmaceutically-acceptable salt thereof, to the patient,
wherein the patient has received, is receiving, or is scheduled to receive the Her2-targeted therapeutic agent.

Embodiment 2. A method of treating a patient with Her2⁺ cancer resistant to a Her2-targeted therapeutic agent comprising administering a RXR agonist of Formula I,

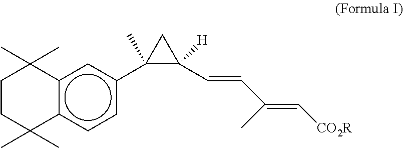

wherein R is H, or lower alkyl of 1 to 6 carbons; or a pharmaceutically-acceptable salt thereof, and the Her2-targeted therapeutic agent.

Embodiment 3. A method of treating a patient with Her2⁺ cancer undergoing treatment with a RXR agonist of Formula I,

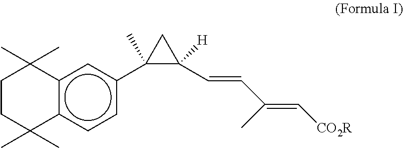

wherein R is H, or lower alkyl of 1 to 6 carbons; or a pharmaceutically-acceptable salt thereof,
wherein the cancer has become resistant to the RXR agonist, comprising continuing treatment with the RXR agonist and initiating treatment with a Her2-targeted therapeutic agent.

Embodiment 4. A method of treating a patient with Her2⁺ cancer undergoing treatment with a Her2-targeted therapeutic agent, wherein the cancer has become resistant to the Her2-targeted therapeutic agent, comprising continuing treatment with the Her2-targeted therapeutic agent and initiating treatment with a RXR agonist of Formula I,

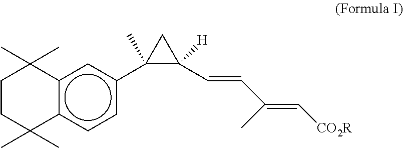

wherein R is H, or lower alkyl of 1 to 6 carbons; or a pharmaceutically-acceptable salt thereof.

Embodiment 5. A method of treating a patient with Her2⁺ cancer resistant to a Her2-targeted therapeutic agent comprising administering a RXR agonist of Formula I,

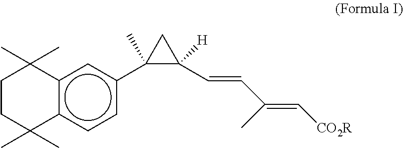

wherein R is H, or lower alkyl of 1 to 6 carbons; or a pharmaceutically-acceptable salt thereof, to the patient,
wherein the patient has received, is receiving, or is scheduled to receive means for therapeutically targeting Her2.

Embodiment 6. The method of Embodiment 5, wherein the means for therapeutically targeting Her2 are:
means for inhibiting Her2⁺ tumor cell proliferation,
means for mediating ADCC of Her2⁺ tumor cells,
immunoglobulin means for inhibiting Her2 signaling,
means for delivering a cytotoxic agent to Her2⁺ cells, or
small molecule means for inhibiting Her2 kinase activity.

Embodiment 7. A method of treating a patient with Her2⁺ cancer resistant to a Her2-targeted therapeutic agent comprising administering means for activating RXR/Nurr1 heterodimeric receptors or rexinoid means for inhibiting tumor growth, wherein the patient has received, is receiving, is scheduled to receive a Her2-targeted therapeutic agent.

Embodiment 8. The method of any one of Embodiments 1-7, further comprising administering thyroid hormone in conjunction with the RXR agonist.

Embodiment 9. The method of Embodiment 8, wherein the thyroid hormone is thyroxine.

Embodiment 10. The method of any one of Embodiments 1-9, wherein the RXR agonist, the means for activating RXR/Nurr1 heterodimeric receptors, or the rexinoid means for inhibiting tumor growth, is a compound of Formula I.

Embodiment 11. The method of any one of Embodiments 1-9, wherein the RXR agonist, the means for activating RXR/Nurr1 heterodimeric receptors, or the rexinoid means for inhibiting tumor growth, is a pharmaceutically-acceptable salt of a compound of Formula I.

Embodiment 12. The method of any one of Embodiments 1-9, wherein the RXR agonist, the means for activating RXR/Nurr1 heterodimeric receptors, or the rexinoid means for inhibiting tumor growth, is a compound of Formula II

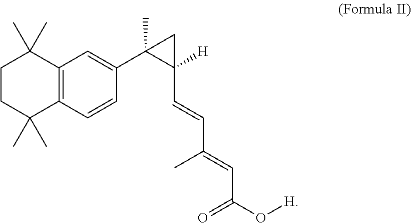

Embodiment 13. The method of any one of Embodiments 1-12, wherein:
the Her2-targeted therapeutic agent,
the means for inhibiting Her2⁺ tumor cell proliferation,
the means for mediating ADCC of Her2⁺ tumor cells,
the immunoglobulin means for inhibiting Her2 signaling, or
the means for delivering a cytotoxic agent to Her2⁺ cells,
comprises an anti-Her2 therapeutic antibody.

Embodiment 14. The method of Embodiment 13, wherein the therapeutic antibody is trastuzumab or pertuzumab.

Embodiment 15. The method of Embodiment 13, wherein the therapeutic antibody is margetuximab, TrasGEX, HM2, hertuzumab, or HT-19

Embodiment 16. The method of any one of Embodiments 1-13, wherein the Her2-targeted therapeutic agent, the means for inhibiting Her2⁺ tumor cell proliferation, or the immunoglobulin means for inhibiting Her2 signaling, comprises an antibody-drug conjugate wherein the antibody is an anti-Her2 antibody.

Embodiment 17. The method of Embodiment 16, wherein the antibody-drug conjugate or the means for delivering a cytotoxic agent to Her2⁺ cells, is ado-trastuzumab emtansine.

Embodiment 18. The method of Embodiment 16, wherein the antibody-drug conjugate or the means for delivering a cytotoxic agent to Her2⁺ cells, is A166, ALT-P7, ARX788, DHES0815A, DS-8201a, RC48, SYD985, MEDI4276, or XMT-1522.

Embodiment 19. The method of any one of Embodiments 1-12, wherein the Her2-targeted therapeutic agent, or the small molecule means for inhibiting Her2 kinase activity, comprises a Her2 kinase inhibitor.

Embodiment 20. The method of Embodiment 19, wherein the Her2 kinase inhibitor is lapatinib or neratinib.

Embodiment 21. The method of Embodiment 19, wherein the Her2 kinase inhibitor is afatinib or dacomitinib.

Embodiment 22. The method of any one of Embodiments 1-21, wherein the treatment is applied as a debulking therapy.

Embodiment 23. The method of any one of Embodiments 1-21, wherein the treatment is applied as adjuvant therapy.

Embodiment 24. The method of any one of Embodiments 1-23, wherein the Her2⁺ cancer is Her2⁺ breast cancer.

Embodiment 25. The method of any one of Embodiments 1-23, wherein the Her2⁺ cancer is Her2⁺ gastroesophageal cancer, ovarian cancer, stomach cancer, adenocarcinoma of the lung, uterine cancer (such as serous endometrial carcinoma), or salivary duct carcinoma.

Embodiment 26. The method of any one of Embodiments 1-25, wherein a therapeutic response to the RXR agonist and the Her2-targeted therapeutic agent is greater than to the response to either of the agents alone.

Embodiment 27. The method of Embodiment 26, wherein the greater therapeutic response is a slowing of tumor growth, stabilization of disease, a partial response, a complete response, extended overall survival, or disease-free survival.

Embodiment 28. The method of Embodiment 26 or 27, wherein response is evaluated according to RECIST or iRECIST criteria.

Embodiment 29. The method of any one of Embodiments 26-28, comprising a reduction or amelioration of secondary symptoms.

Embodiment 30. The method of any one of Embodiments 26-29, wherein the Her2⁺ cancer resistant to a Her2-targeted therapeutic agent is also resistant to the RXR agonist.

Embodiment 31. A RXR agonist of Formula I,

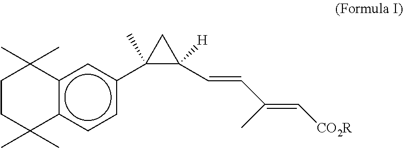

wherein R is H, or lower alkyl of 1 to 6 carbons; or a pharmaceutically-acceptable salt thereof, for use in manufacturing a medicament for treating Her2⁺ cancer resistant to a Her2-targeted therapeutic agent, in a patient who has received, is receiving, or is scheduled to receive a Her2-targeted therapeutic agent.

Embodiment 32. A RXR agonist of Formula I,

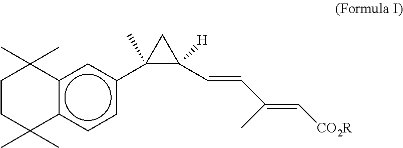

wherein R is H, or lower alkyl of 1 to 6 carbons; or a pharmaceutically-acceptable salt thereof, and a Her2-targeted therapeutic agent, for use in manufacturing medicaments for use together to treat Her2⁺ cancer resistant to a Her2-targeted therapeutic agent.

Embodiment 33. A RXR agonist of Formula I,

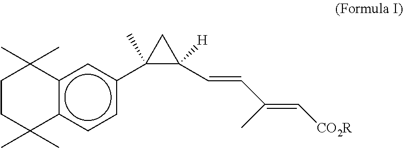

wherein R is H, or lower alkyl of 1 to 6 carbons; or a pharmaceutically-acceptable salt thereof, for treating Her2⁺ cancer resistant to a Her2-targeted therapeutic agent in a patient with Her2⁺ cancer,
in combination with the Her2-targeted therapeutic agent.

It should be manifest that each or Embodiments 31-33 can be modified in a manner similar to the modification of Embodiments 1-5 and 7 by Embodiments 5 and 8-29.

### EXAMPLES

The following non-limiting examples are provided for illustrative purposes only in order to facilitate a more complete understanding of representative embodiments now contemplated. These examples should not be construed to limit any of the embodiments described in the present specification.

### Example 1

### Inhibition of Breast Cancer Cell Growth by IRX4204 Plus Trastuzumab

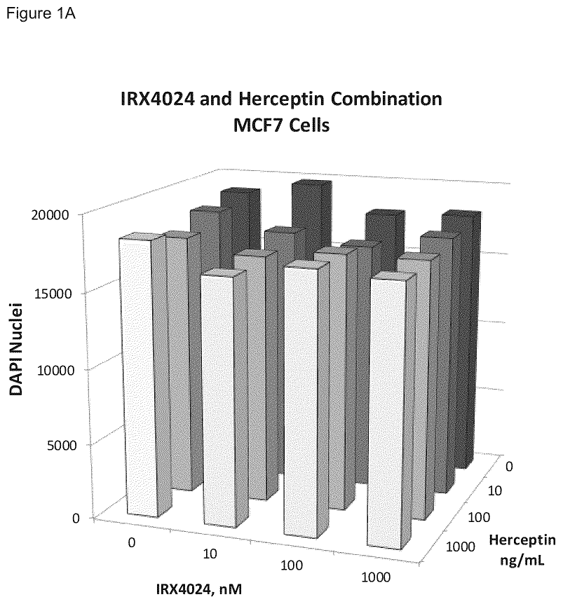

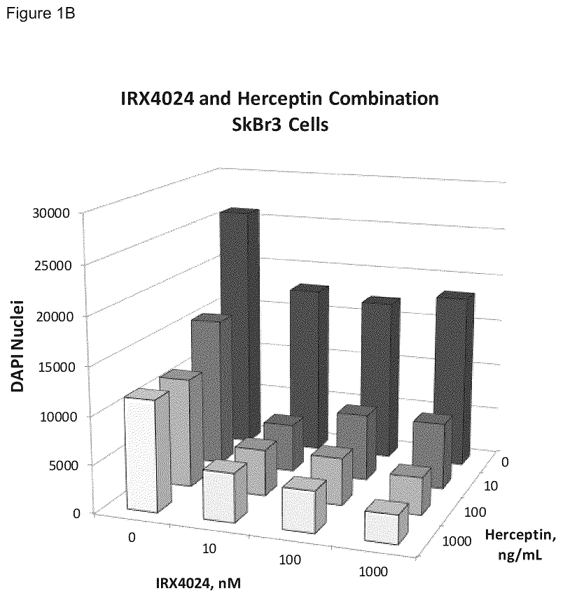

Two breast cancer cell lines, MCF7 and SkBr3, were cultured in the presence of 0, 10, 100, or 1000 nM IRX4204 and 0, 0.1, 1, or 10 μg/ml trastuzumab. MCF7 is an ER⁺ PR⁺ Her2⁻ cell line. SkBr3 is an ER⁻ PR⁻ Her2⁺ cell line. Cells were plated in 96 well optical plates and IRX-4204 and trastuzumab were added 24 hours after cell plating. After a further 6 days, the cells were fixed with 4% paraformaldehyde in phosphate buffered saline (PBS). Nuclei were then stained with DAPI and imaged with the MetaXpress® microscope (Molecular Devices, San Jose, CA). Cell nuclei were segmented and then counted by defining pixel intensity over background and object size, using the algorithm of the MetaXpress® image analysis software. Experimental data points were performed at a minimum of quadruplicate, and results were reported as average cell count ±standard deviation (SD). As seen in FIG. 1A, neither agent had more than marginal effect on the Her2⁻ MCF7 cell line. As seen in FIG. 1B, both agents individually have a moderate growth inhibitory effect on the Her2⁺ SkBr3 cell line, and had a very substantial, growth inhibitory effect when used in combination, even at the lowest concentrations tested.

To assess if this improved growth inhibition effect was synergistic, the percent inhibition observed for the highest concentrations of the combined therapeutic agents was compared to the percent inhibition that would be expected for the combination based on the observed inhibition of the therapeutic agents used alone, if the agents acting independently, that is, without interaction (see Table 1). That is, PAE=(FE1+((1−FE1)×FE2))×100, where PAE is the predicted additive effect, FE1 is the observed fractional effect of a 1st treatment, and FE2 is the observed fractional effect of a 2nd treatment.

TABLE 1
Synergistic Effect of the Combination of IRX4204 and Trastuzumab
on the Inhibition of the Growth of SkBr3 Cells.

| Treatment | DAPI Nuclei Count | % Inhibition | |
|---|---|---|---|
| Control | 25958 | | |
| IRX4204 1000 nM | 18274 | 29.4 | If Additive = |
| Herceptin 1000 ng | 11582 | 55.4 | 68.5% |
| IRX4204 + Herceptin | 2946 | 88.7 | |

Whereas the combined inhibitory effect of IRX4204 and trastuzumab would be predicted to be 68.5% if there was no interaction between the effects of the two agents, in fact the observed degree of inhibition was 88.7%, clearly indicating that IRX4204 and trastuzumab interact in a synergistic manner.

### Example 2

### Inhibition of Breast Cancer Cell Growth by IRX4204 Plus Lapatinib or Neratinib

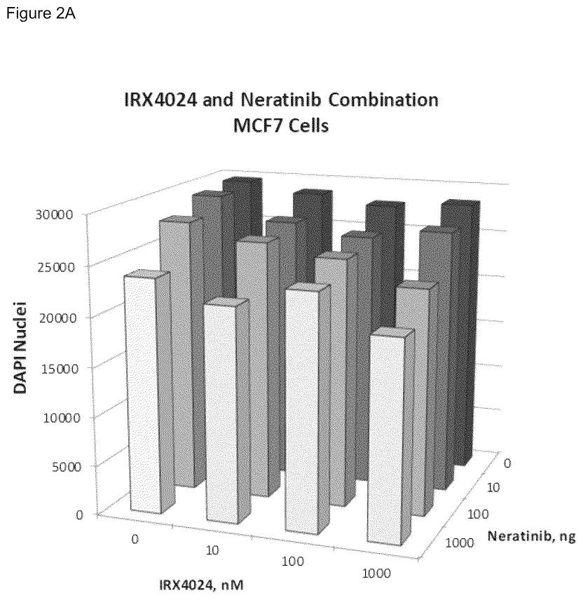

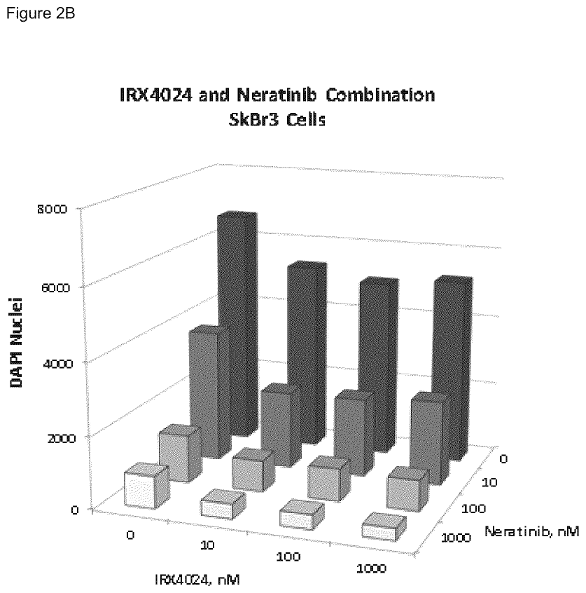

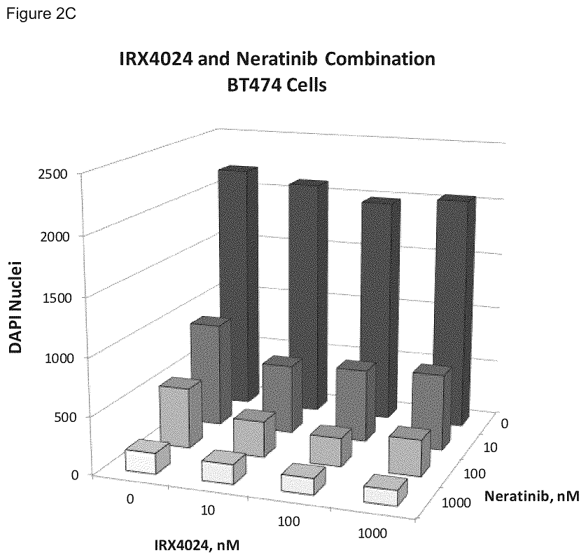

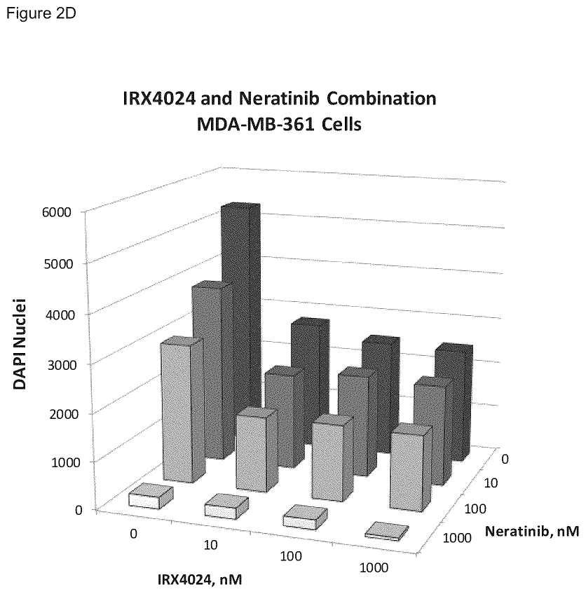

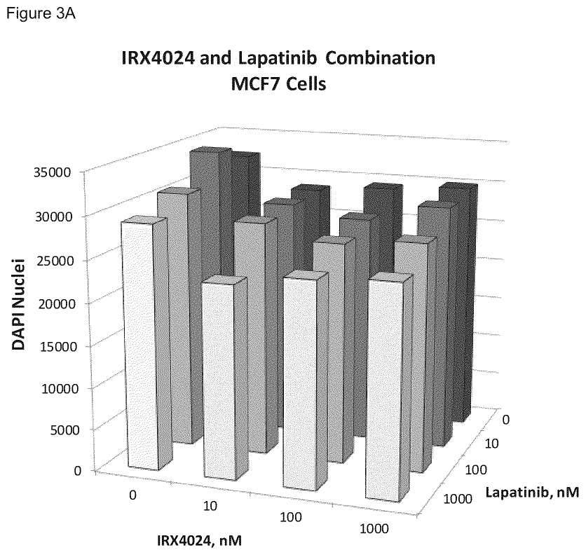

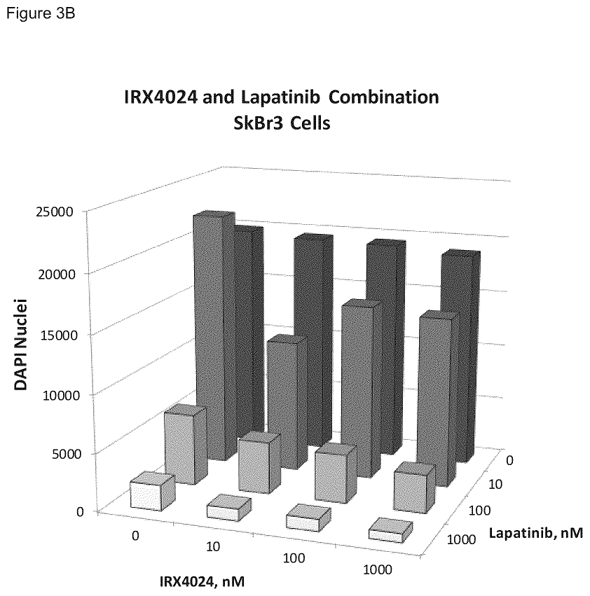

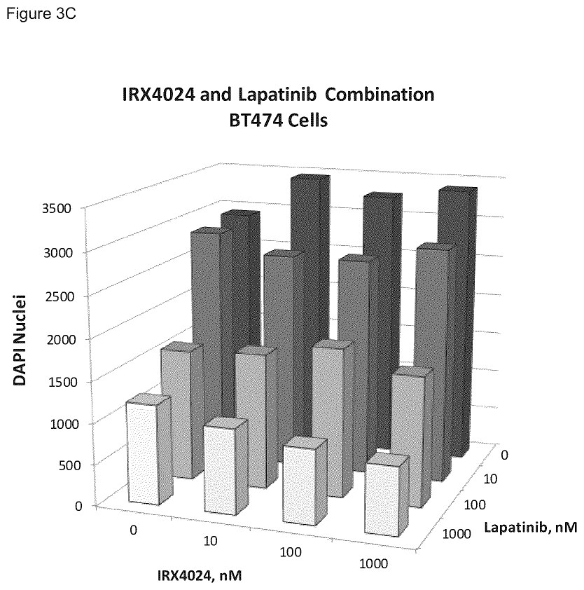

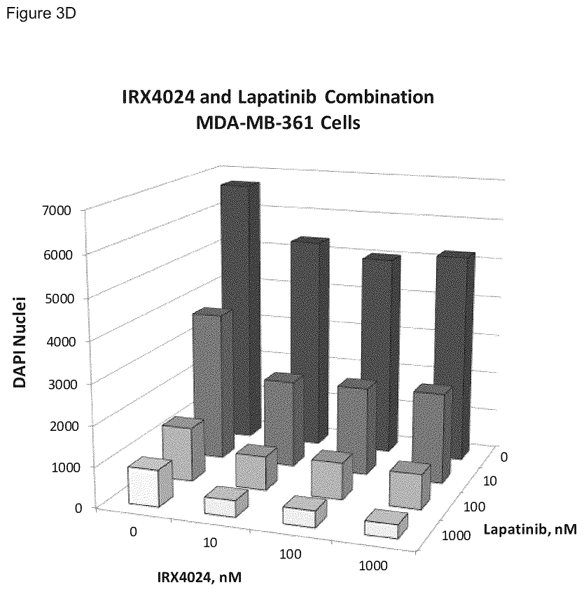

The experiment of Example 1 was repeated using the Her2 kinase inhibitors lapatinib or neratinib instead of trastuzumab at 0, 0.1, 1, or 10 nM. Additionally the panel of breast cancer cell lines was expanded to also include the ER⁺ PR⁺ Her2⁺ cell line BT474 and the ER⁺ PR⁻ Her2⁺ cell line MDA-MB-361. The general pattern seen above, that the Her2⁻ MCF7 cell line showed generally marginal response to the treatments and that the Her⁺ cell lines exhibited a greater degree of inhibition to the combination than either agent alone, was again observed (see FIGS. 2 and 3).

To assess if this improved growth inhibition effect was synergistic, the percent inhibition observed for the highest concentrations of the combined therapeutic agents was compared to the percent inhibition that would be expected for the combination based on the observed inhibition of the therapeutic agents used alone, if the agents acting independently, that is, without interaction (see Tables 2-4).

TABLE 2
Synergistic Effect of the Combination of IRX4204 and Lapatinib
or Neratinib on the Inhibition of the Growth of SkBr3 Cells.

| Treatment | DAPI Nuclei Count | % Inhibition | |
|---|---|---|---|
| Control | 19855 | | |
| IRX4204 1000 nM | 18906 | 4.8 | If Additive = |
| Lapatinib 1000 nM | 2211 | 88.9 | 89.4% |
| IRX4204 + Lapatinib | 743 | 96.3 | |
| Control | 6696 | | |
| IRX4204 1000 nM | 5218 | 22.1 | If Additive = |
| Neratinib 1000 nM | 903 | 86.5 | 89.5% |
| IRX4204 + Neratinib | 329 | 95.1 | |

TABLE 3
Synergistic Effect of the Combination of IRX4204 and Lapatinib
or Neratinib on the Inhibition of the Growth of BT474 Cells.

| Treatment | DAPI Nuclei Count | % Inhibition | |
|---|---|---|---|
| Control | 2937 | | |
| IRX4204 1000 nM | 3399 | −15.7* | If Additive = |
| Lapatinib 1000 nM | 1211 | 58.8 | 58.8% |
| IRX4204 + Lapatinib | 813 | 72.3 | |
| Control | 2192 | | |
| IRX4204 1000 nM | 2043 | 6.8 | If Additive = |
| Neratinib 1000 nM | 180 | 91.8 | 92.4% |
| IRX4204 + Neratinib | 130 | 94.1 | |

*Zero inhibition used in calculation of predicted additive effect

TABLE 4
Synergistic Effect of the Combination of IRX4204 and Lapatinib or
Neratinib on the Inhibition of the Growth of MDA-MB-361 Cells.

| Treatment | DAPI Nuclei Count | % Inhibition | |
|---|---|---|---|
| Control | 6696 | | |
| IRX4204 1000 nM | 5218 | 22.1 | If Additive = |
| Lapatinib 1000 nM | 903 | 86.5 | 89.5% |
| IRX4204 + Lapatinib | 329 | 95.2 | |
| Control | 5297 | | |
| IRX4204 1000 nM | 2459 | 43.6 | If Additive = |
| Neratinib 1000 nM | 249 | 95.3 | 97.3% |
| IRX4204 + Neratinib | 57 | 98.9 | |

In each case the observed degree of inhibition exceeded that predicted if there was no interaction between the effects of the two agents, even though in some cases the individual therapeutic agents were quite effective alone, leaving little room for synergy to be observed. These data also clearly indicate that IRX4204 and the small molecule Her2 kinase inhibitors interact in a synergistic manner.

### Example 3

### Inhibition of Growth of a Her2-Targeted Therapeutic Agent Resistant Her2⁺ Cancer Cell Line

HCC1954 is Her2⁺ cancer cell line derived from a ductal carcinoma of the breast. As compared to the SkBr3 cell line used in Example 1 above, it is resistant to neratinib, lapatinib, and tucatinib. It is also resistant to anti-Her2 monoclonal antibodies, such as trastuzumab. Additionally, it is highly resistant to IRX4204. Among Her2-amplified cell lines, HCC1954 is the most resistant to neratinib with an IC50>100 nM (compared to 7 nM SkBr3 or 20 nM AU565).

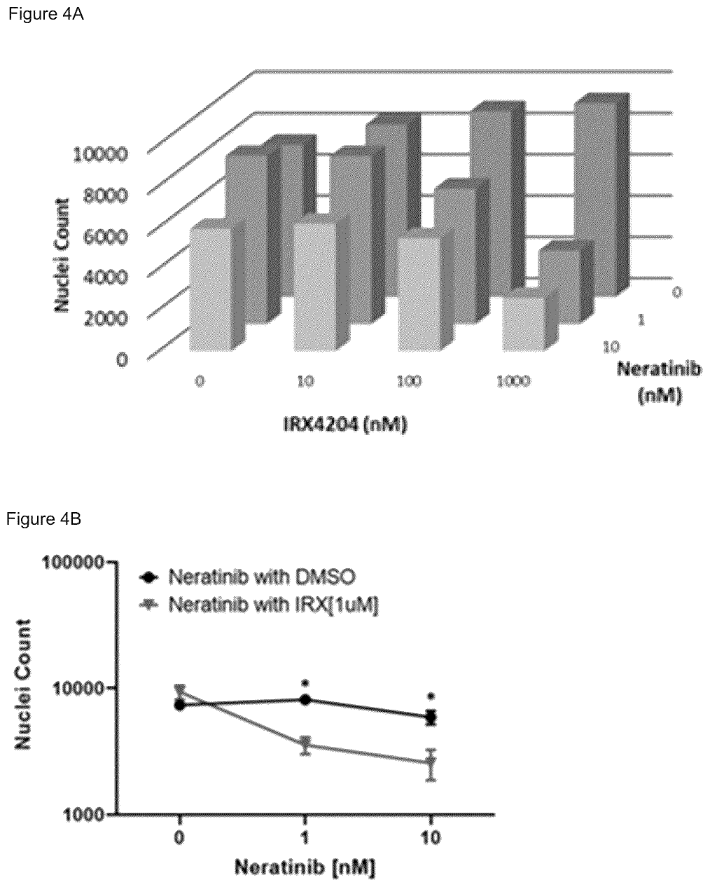

HCC1954 human breast cancer cells were seeded at 3000 cells per well in 96-well plates and treated with neratinib [1 or 10 nM] or DMSO (0.1%) in combination with IRX4204 [10, 100, or 1000 nM]. After 6 days of treatment, cells were fixed with 4% paraformaldehyde. Nuclei were stained with DAPI, imaged using ImageXpress® Pico (Molecular Devices) and counted using CellReporterXpress® Analysis Software (FIGS. 4A-B) Each data point represents six replicates. Statistical significance was determined using the Bonferroni-Dunn method of multiple t-tests. Each day was analyzed individually, without assuming a consistent SD (*p<0.01). As IRX4204 had no growth inhibitory effect on this cell line as a sole agent, the improved inhibition when used in combination with neratinib is a clear synergistic effect.

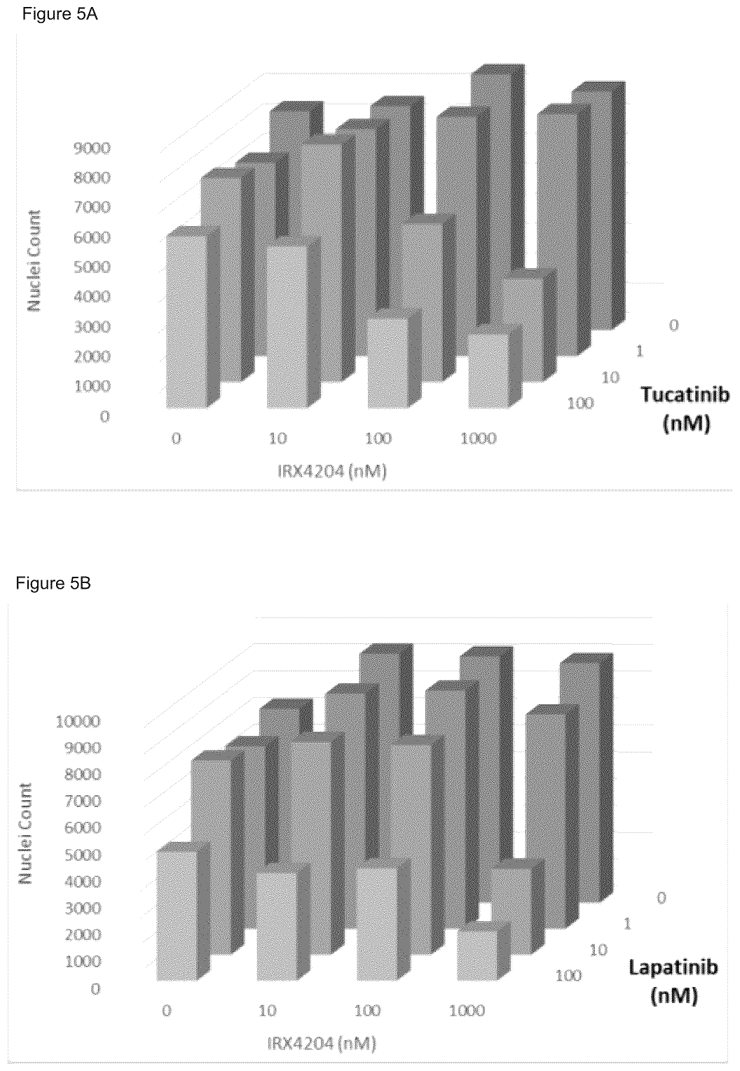

The experiment was also conducted using tucatinib or lapatinib in place of neratinib. Tucatinib has an IC50>2 μM on HCC1954 as compared to 22 nM on SkBr3 or 125 nM on AU565. Lapatinib has an IC50>1.4 μM on HCC1954 as compared to 152 nM on SkBr3 or 294 nM on AU565. The same general pattern of inhibitory effect is observed as with neratinib. Namely, the IRX4204 had no growth inhibitory effect on this resistant cell line, the kinase inhibitor has some small effect, but at the higher concentrations tested, the combination of the RXR agonist and the Her2 kinase inhibitor had a profound growth inhibitory effect (FIG. 5A-B). As with neratinib, there is clearly a synergistic effect from the combination of these kinase inhibitors with IRX4204.

While there are some common elements in the structures of these three kinase inhibitors, they are not close structural analogs. Thus, the synergy observed between these three kinase inhibitors and IRX4204 supports the generality of the effect with respect to Her2 kinase inhibitors.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a patient with Her2⁺ cancer resistant to at least one Her2-targeted therapeutic agent, the method comprising administering a retinoid X receptor (RXR) agonist of Formula I 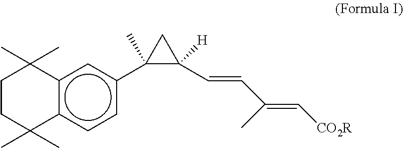
wherein R is H, or lower alkyl of 1 to 6 carbons, or a pharmaceutically-acceptable salt thereof, to the patient,
wherein the patient has received or is receiving the Her2-targeted therapeutic agent.

2. The method of claim 1, further comprising administering thyroid hormone in conjunction with the RXR agonist.

3. The method of claim 1, wherein the RXR agonist is a compound of Formula II

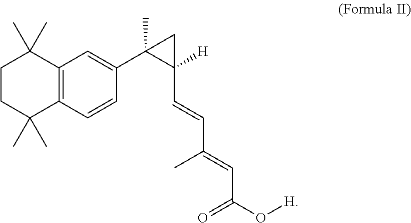

4. The method of claim 1, wherein the Her2-targeted therapeutic agent comprises an anti-Her2 therapeutic antibody.

5. The method of claim 4, wherein the therapeutic antibody is trastuzumab or pertuzumab.

6. The method of claim 1, wherein the Her2-targeted therapeutic agent comprises an antibody-drug conjugate wherein the antibody is an anti-Her2 antibody.

7. The method of claim 1, wherein the Her2-targeted therapeutic agent comprises a Her2 kinase inhibitor.

8. The method of claim 7, wherein the Her2 kinase inhibitor comprises lapatinib, neratinib, tucatinib, afatinib, dacomitinib, canertinib, sapitinib, CP-724714, or CUDC-101.

9. The method of claim 1, wherein the Her2⁺ cancer is Her2⁺ breast cancer.

10. The method of claim 1, wherein tumor cell growth is inhibited by a combination of the Her2-targeted therapeutic and the RXR agonist, and the inhibition of tumor cell growth by the combination is greater than additive inhibitory effects of each of the Her2-targeted therapeutic and the RXR agonist alone.

11. A method of treating a patient with Her2⁺ cancer undergoing treatment with a Her2-targeted therapeutic agent, wherein the cancer has become resistant to the Her2-targeted therapeutic agent, the method comprising continuing treatment with the Her2-targeted therapeutic agent and initiating treatment with an RXR agonist of Formula I, 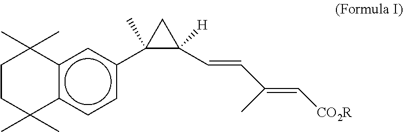
wherein R is H, or lower alkyl of 1 to 6 carbons, or a pharmaceutically-acceptable salt thereof.

12. The method of claim 11, further comprising administering thyroid hormone in conjunction with the RXR agonist.

13. The method of claim 11, wherein the RXR agonist is a compound of Formula II

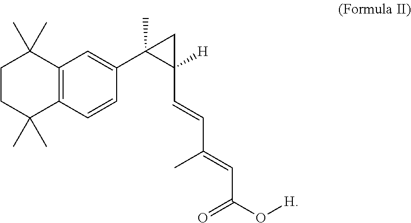

14. The method of claim 11, wherein the Her2-targeted therapeutic agent comprises an anti-Her2 therapeutic antibody.

15. The method of claim 14, wherein the therapeutic antibody is trastuzumab or pertuzumab.

16. The method of claim 14, wherein the Her2-targeted therapeutic agent comprises an antibody-drug conjugate wherein the antibody is an anti-Her2 antibody.

17. The method of claim 11, wherein the Her2-targeted therapeutic agent comprises a Her2 kinase inhibitor.

18. The method of claim 17, wherein the Her2 kinase inhibitor comprises lapatinib, neratinib, tucatinib, afatinib, dacomitinib, canertinib, sapitinib, CP-724714, or CUDC-101.

19. The method of claim 13, wherein the Her2⁺ cancer is Her2⁺ breast cancer.

20. The method of claim 13, wherein tumor cell growth is inhibited by a combination of the Her2-targeted therapeutic and the RXR agonist, and the inhibition of tumor cell growth by the combination is greater than additive inhibitory effects of each of the Her2-targeted therapeutic and the RXR agonist alone.
